(19) United States
(12) Patent Application Publication
(10) Pub. No.: US 2016/0340286 A1
(43) Pub. Date: Nov. 24, 2016

(54) CLOSTRUBINS

(71) Applicant: LEIBNIZ-INSTITUT FÜR NATURSTOFF-FORSCHUNG UND INFEKTIONSBIOLOGIE E.V., Jena (DE)

(72) Inventors: Sacha Pidot, Mooroolbark (AU); Keishi Ishida, Jena (DE); Gulimila Shabuer, Jena (DE); Michael Cyrulies, Leipzig (DE); Christian Hertweck, 04105 Leipzig (DE)

(21) Appl. No.: 15/114,752

(22) PCT Filed: Jan. 28, 2015
(86) PCT No.: PCT/EP2015/000162

§ 371 Date: Jul. 27, 2016

(30) Foreign Application Priority Data
Jan. 28, 2014 (EP) 14000306.2
Feb. 3, 2014 (GB) 1401807.1

(51) Int. Cl. C07C 49/747; C12P 15/00

(52) U.S. Cl. CPC C07C 49/747; C12P 15/00; C07C 2103/52

(57) ABSTRACT

The present invention relates to a bioactive compound according to general formula (I); to a pharmaceutical composition comprising one or more of the compound(s); and to the use of the compound(s) as an antibiotic, cytotoxic and/or anti—proliferative agent.

### FIELD OF THE INVENTION

The present invention relates to a bioactive compound according to general formula (I); to a pharmaceutical composition comprising one or more of the compound(s); and to the use of the compound(s) as an antibiotic, cytotoxic and/or anti-proliferative agent.

### BACKGROUND OF THE INVENTION

In view of the rapid decline in the effectiveness of antibiotics due to the emergence of resistance, there is a need for a constant supply of new antibiotics for effective treatment of bacterial infections.

Similarly, the treatment of proliferative diseases such as cancer is one of the biggest medicinal challenges, since these diseases are one of the leading causes of death. Although much progress has been made in the development of anti-proliferative agents, there is a need for novel agents that help expanding the life expectancy of patients suffering from a proliferative disease.

Therefore, the problem underlying the present invention is to provide novel compounds having antibacterial, cytotoxic and/or anti-proliferative activity, especially compounds having antimicrobial activity against Gram-positive bacteria.

### SUMMARY AND DESCRIPTION OF THE INVENTION

The present invention relates to a compound of the general formula (I):

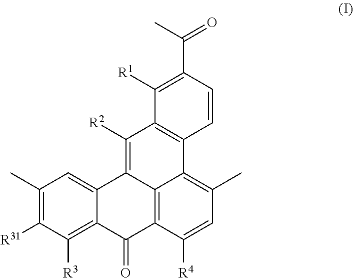

or a pharmacologically acceptable salt thereof, wherein

R¹, R², R³, R⁴ and R³¹ each independently represents H, OH, F, Cl, Br, I, NO₂, CF₃, CN, NH₂, NO₂, —(CH₂)ₘ—R; —C(═O)R; —C(═O)NHR; —NHC(═O)R; —NHSO₂R; S(O)ₙX²; SO₂NR⁵R⁶; or a group comprising a linear or branched polyethylene glycol (PEG) group which comprises the formula —(CH₂—CH₂—O)ₜ— with t being an integer of from 2 to 100 and has a molecular weight (MW) of from 400 to 50000 Da;

R represents H; —(CH₂)ₚ—X^{l}; —(CH₂)ₚ—OX¹; a C₁₋₆ heteroalkyl; a cycloalkyl; a heterocycloalkyl; an alkylcycloalkyl; a heteroalkylcycloalkyl; an aryl; a heteroaryl; an aralkyl; or a heteroaralkyl group;

R⁵ and R⁶ each independently represents a hydrogen atom; or —(CH₂)ₘ—R; or

R⁵ and R⁶ are taken together to form a 5-to 8-membered saturated, unsaturated or aromatic heterocycle containing 1 to 4 N atoms or 1 to 3 heteroatoms selected from the group consisting of nitrogen, oxygen and sulphur, which heterocycle may be unsubstituted or mono-, di or trisubstituted by halogen atom or R; or R⁵ and R⁶ are taken together to form a 5-to 8-membered saturated, unsaturated or aromatic heterocycle containing 1 to 3 heteroatoms selected from the group consisting of nitrogen, oxygen and sulphur, which is fused to one or two rings selected from the group consisting of cycloalkyl; heterocycloalkyl; alkylcycloalkyl; heteroalkylcycloalkyl; aryl; heteroaryl; aralkyl; and heteroaralkyl;

X¹ represents a C₁₋₆ alkyl, C₂₋₆ alkenyl, or C₂₋₆ alkynyl group, in which 1 to 5 H atoms may, independently of each other, be replaced by halogen atom, CN, CF₃, NO₂, OR or NHR, and/or in which one or two non-adjacent CH₂ group(s) may be replaced by O, NH, S, SO, SO₂, or C₃₋₇ cycloalkyl;

X² represents CN, CF₃, —(CH₂)ₚ—X¹, or NR⁵R⁶;

m is 0, 1, 2 or 3;

n is 0, 1 or 2; and

p is an integer from 0 to 6.

Compounds are usually described herein using standard nomenclature or the definitions presented below. For compounds having asymmetric centers, it should be understood that (unless otherwise specified) all of the optical isomers and mixtures thereof are encompassed. Compounds with two or more asymmetric elements can also be present as mixtures of diastereomers. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope (i.e., an atom having the same atomic number but a different mass number). By way of general example, and without limitation, isotopes of hydrogen include tritium and deuterium and isotopes of carbon include ¹¹ C, ¹³C, and ¹⁴C.

Compounds according to the formulas provided herein, which have one or more stereogenic centers, have an enantiomeric excess of at least 50%. For example, such compounds may have an enantiomeric excess of at least 60%, 70%, 80%, 85%, 90%, 95%, or 98%. Some embodiments of the compounds have an enantiomeric excess of at least 99%. It will be apparent that single enantiomers (optically active forms) can be obtained by asymmetric synthesis, synthesis from optically pure precursors or by resolution of the racemates. Resolution of the racemates can be accomplished, for example, by conventional methods such as crystallization in the presence of a resolving agent, or chromatography, using, for example a chiral HPLC column.

Certain compounds are described herein using a general formula that includes variables, e.g. R¹, R², R³, and R⁴. Unless otherwise specified, each variable within such a formula is defined independently of any other variable, and any variable that occurs more than one time in a formula is defined independently at each occurrence. Thus, for example, if a group is shown to be substituted with 0-2 R^{*}, the group may be unsubstituted or substituted with up to two R^{*} groups and R^{*} at each occurrence is selected independently from the definition of R^{*}. Also, combinations of substituents and/or variables are permissible only if such combinations result in stable compounds, i.e., compounds that can be isolated, characterized and tested for biological activity.

As used herein a wording defining the limits of a range of length such as, e. g., “from 1 to 5” means any integer from 1 to 5, i. e. 1, 2, 3, 4 and 5. In other words, any range defined by two integers explicitly mentioned is meant to comprise and disclose any integer defining said limits and any integer comprised in said range. For example, the term “C₁-C₆” refers to 1 to 6, i.e. 1, 2, 3, 4, 5 or 6, carbon atoms.

A “pharmacologically acceptable salt” of a compound disclosed herein is an acid or base salt that is generally considered in the art to be suitable for use in contact with the tissues of human beings or animals without excessive toxicity or carcinogenicity, and preferably without irritation, allergic response, or other problem or complication. Such pharmaceutical salts include mineral and organic acid salts of basic residues such as amines, as well as alkali or organic salts of acidic residues such as carboxylic acids.

Suitable pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is any integer from 0 to-4 (i.e., 0, 1, 2, 3, or 4) and the like. Similarly, pharmaceutically acceptable cations include, but are not limited to sodium, potassium, calcium, aluminum, lithium and ammonium. Those of ordinary skill in the art will recognize further pharmacologically acceptable salts for the compounds provided herein. In general, a pharmacologically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Generally, the use of nonaqueous media, such as ether, ethyl acetate, ethanol, isopropanol or acetonitrile, is preferred.

It will be apparent that each compound of formula (I) may, but need not, be present as a hydrate, solvate or non-covalent complex. In addition, the various crystal forms and polymorphs are within the scope of the present invention, as are prodrugs of the compounds of formula (I) provided herein.

A “prodrug” is a compound that may not fully satisfy the structural requirements of the compounds provided herein, but is modified in vivo, following administration to a subject or patient, to produce a compound of formula I provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

A “substituent,” as used herein, refers to a molecular moiety that is covalently bonded to an atom within a molecule of interest. For example, a “ring substituent” may be a moiety such as a halogen, alkyl group, haloalkyl group, hydroxy, cyano, amino, nitro, mercapto, or other substituent described herein that is covalently bonded to an atom (preferably a carbon or nitrogen atom) that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e. a compound that can be isolated, characterized and tested for biological activity. When a substituent is oxo (i.e., ═0), then 2 hydrogens on the atom are replaced. An oxo group that is a substituent of an aromatic carbon atom results in a conversion of —CH—to —C(═O)—and a loss of aromaticity. For example a pyridyl group substituted by oxo is a pyridone.

As used herein, “comprising”, “including”, “containing”, “characterized by”, and grammatical equivalents thereof are inclusive or open—ended terms that do not exclude additional, unrecited elements or method steps. “Comprising”, etc. is to be interpreted as including the more restrictive term “consisting of”.

As used herein, “consisting of” excludes any element, step, or ingredient not specified in the claim.

When trade names are used herein, it is intended to independently include the trade name product formulation, the generic drug, and the active pharmaceutical ingredient(s) of the trade name product.

In general, unless defined otherwise, technical and scientific terms used herein have the same meaning as commonly understood by one of ordinary skill in the art to which this invention belongs, and are consistent with general textbooks and dictionaries.

The expression “optionally substituted” refers to groups in which one or more hydrogen atoms have been replaced each independently of the others by hydrogen, fluorine, chlorine, bromine or iodine atoms or by OH, ═O, SH, ═S, NH₂, ═NH, CN or NO₂ groups. This expression refers furthermore to groups in which one or more hydrogen atoms have been replaced each independently of the others by unsubstituted C₁-C₆alkyl, (C₁-C₆) haloalkyl (e.g. a fluoromethyl, trifluoromethyl, chloromethyl, (1- or 2-)haloethyl (e.g. (1- or 2-) chloroethyl), or (2- or 3-) halopropyl (e.g. (2- or 3-) fluoropropyl) group), (C₁-C₆) hydroxyalkyl (e.g. a hydroxymethyl, (1- or 2-)hydroxyethyl, or (2- or 3-) hydroxypropyl group), unsubstituted C₂-C₆alkenyl, unsubstituted C₂-C₆alkynyl, unsubstituted C₁-C₆heteroalkyl, unsubstituted C₃-C₁₀cycloalkyl, unsubstituted C₂-C₉heterocycloalkyl, unsubstituted C₆-C₁₀aryl, unsubstituted C₁-C₉heteroaryl, unsubstituted C₇-C₁₂aralkyl or unsubstituted C₂-C₁ᵢheteroaralkyl groups.

The expression alkyl refers to a saturated, straight-chain or branched hydrocarbon group that contains from 1 to 20 carbon atoms, preferably from 1 to 12 carbon atoms, more preferably from 1 to 6 carbon atoms, for example a methyl, ethyl, propyl, iso-propyl, n-butyl, iso-butyl, sec-butyl, tert-butyl, n-pentyl, n-hexyl, 2,2-dimethylbutyl or n-octyl group. An alkyl group may optionally be substituted.

The expression alkenyl refers to an at least partially unsaturated, straight-chain or branched hydrocarbon group that contains one or more double bond(s) and from 2 to 20 carbon atoms, preferably from 2 to 12 carbon atoms, more preferably from 2 to 6 carbon atoms, for example an ethenyl (vinyl), propenyl (allyl), iso-propenyl, butenyl, isoprenyl or hex-2-enyl group. Preferably, an alkenyl group has one or two, especially one, double bond(s).

The expression alkynyl refers to an at least partially unsaturated, straight-chain or branched hydrocarbon group that contains one or more triple bond(s) and from 2 to 20 carbon atoms, preferably from 2 to 12 carbon atoms, more preferably from 2 to 6, e.g. 2, 3 or 4, carbon atoms, for example an ethynyl (acetylenyl), propynyl, butynyl or propargyl group. Preferably, an alkynyl group has one or two, especially one, triple bond(s).

As used herein, the expression “heteroalkyl” also includes heteroalkenyl and heteroalkynyl, and accordingly refers to an alkyl, alkenyl or alkynyl (straigt chain or branched) group as defined above, in which one or more, preferably 1, 2, 3 or 4, carbon atoms have been replaced each independently of the others by an oxygen, nitrogen, phosphorus, boron, selenium, silicon or sulphur atom, preferably by an oxygen, sulphur or nitrogen atom, or by a SO or SO₂ group. As a result, the expression heteroalkyl also encompasses groups derived from a carboxylic acid, such as, for example, acyl, acylalkyl, alkoxycarbonyl, acyloxy, acyloxyalkyl, carboxyalkylamide or alkoxycarbonyloxy. Examples of heteroalkyl groups are alkylamino, dialkylamino, alkylaminoalkyl, dialkylaminoalkyl, acylalkyl, alkoxycarbonyl, alkylcarbamoyl, alkylamido, alkylcarbamoylalkyl, alkylamidoalkyl, alkylcarbamoyloxyalkyl, alkylureidoalkyl, alkoxycarbonyloxy, alkoxy, or alkoxyalkyl.

Further examples of heteroalkyl groups are groups of formulae: R^{a}—O—Y^{a}—, R^{a}—S—Y^{a}—, R^{a}—N(R^{b})—Y^{a}—, R^{a}—CO—Y^{a}—, R^{a}—O—CO—Y^{a}—, R^{a}—CO—O—Y^{a}—, R^{a}—CO—N(R^{b})—Y^{a}—, R^{a}—N(R^{b})—, CO—Y^{a}—, R^{a}—O—CO—N(R^{b})—Y^{a}—, R^{a}—N(R^{b})—CO—O—Y^{a}—, R^{a}—N(R^{b})—CO—N(R^{c})—Y^{a}—, R^{a}—O—CO—O—Y^{a}—, R^{a}—N(R^{b})—C(═NR^{d})—N(R^{c})—Y^{a}—, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}—, R^{a}—N(R^{b})—CS—N(R^{c})—Y^{a}—, R^{a}—O—CS—O—Y^{a}—, R^{a}—S—CO—Y^{a}—, R^{a}—CO—S—Y^{a}—, R^{a}—S—CO—N(R^{b})—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, wherein R^{a} being a hydrogen atom, a C₁-C₆ alkyl, a C₂-C₆ alkenyl or a C₂-C₆ alkynyl group; R^{b} being a hydrogen atom, a C₁-C₆ alkyl, a C₂-C₆ alkenyl or a C₂-C₆ alkynyl group; R^{c} being a hydrogen atom, a C₁-C₆ alkyl, a C₂-C₆ alkenyl or a C₂-C₆ alkynyl group; R^{d} being a hydrogen atom, a Cᵢ-C₆ alkyl, a C₂-C₆ alkenyl or a C₂-C₆ alkynyl group and Y^{a} being a direct bond, a C₁-C₆ alkylene, a C₂-C₆ alkenylene or a C₂-C₆ alkynylene group. Specific examples of heteroalkyl groups include acyl, methoxy, trifluoromethoxy, ethoxy, n-propyloxy, isopropyloxy, tert-butyloxy, methoxymethyl, ethoxymethyl, methoxyethyl, methylamino, ethylamino, dimethylamino, diethylamino, isopropylethylamino, methylaminomethyl, ethylaminomethyl, diisopropyl-aminoethyl, dimethylaminomethyl, dimethylaminoethyl, acetyl, propionyl, butyryloxy, acetyloxy, methoxycarbonyl, ethoxycarbonyl, isobutyrylamino-methyl, N-ethyl-N-methyl-carbamoyl, N-methylcarbamoyl, cyano, nitrile, isonitrile, thiocyanate, isocyanate, isothiocyanate and alkylnitrile.

The expression cycloalkyl refers to a saturated or partially unsaturated cyclic group that contains one or more rings (preferably 1 or 2), containing from 3 to 14 ring carbon atoms, preferably from 3 to 10 (more preferably 3, 4, 5, 6 or 7) ring carbon atoms. In an embodiment a partially unsaturated cyclic group has one, two or more double bonds, such as a cycloalkenyl group. Specific examples of a cycloalkyl group are a cyclopropyl, cyclobutyl, cyclopentyl, spiro[4,5]decanyl, norbornyl, cyclohexyl, cyclopentenyl, cyclohexadienyl, decalinyl, bicyclo-[4.3.0]nonyl, cyclopentylcyclohexyl, and a cyclohex-2-enyl group.

The expression heterocycloalkyl refers to a cycloalkyl group as defined above in which one or more, preferably 1, 2 or 3, ring carbon atoms have been replaced each independently of the others by an oxygen, nitrogen, or sulphur atom (preferably oxygen or nitrogen), or by a SO or SO2 group. A heterocycloalkyl group has preferably 1 or 2 ring(s) containing from 3 to 10 (more preferably 3, 4, 5, 6 or 7) ring atoms. Examples are a aziridinyl, oxiranyl, thiiranyl, oxaziridinyl, dioxiranyl, azetidinyl, oxetanyl, thietanyl, diazetidinyl, dioxetanyl, dithietanyl, pyrrolidinyl, tetrahydrofuranyl, thiolanyl, phospholanyl, silolanyl, azolyl, thiazolyl, isothiazolyl, imidazolidinyl, pyrazolidinyl, oxazolidinyl, isoxazolidinyl, thiazolidinyl, isothiazolidinyl, dioxolanyl, dithiolanyl, piperazinyl, morpholinyl, thiopmorpholinyl, trioxanyl, azepanyl, oxepanyl, thiepanyl, homopiperazinyl, or urotropinyl group. Further examples are a 2-pyrazolinyl group, and also a lactam, a lactone and a cyclic imide. The heterocycloalkyl group can be optionally substituted, and may be saturated or mono- , di- or tri-unsaturated. As a result, a group derived from a carbohydrate or saccharide, such as furanoses or pentoses, e.g. arabinose, ribose, xylose, lyxose or desoxyribose, or pyranoses/hexoses or derivatives thereof, e.g. allose, altrose, glucose, mannose, gulose, idose, galactose, talose, 6-carboxy-D-glucose, 6-carboxy-D-galactose, N-acetylchitosamine, glucosamine, N-acetylchondrosamin, fucose, rhamnose, chinovose, represents an optionally substituted heterocycloalkyl group.

The expression alkylcycloalkyl refers to a group containing both cycloalkyl and also an alkyl, alkenyl or alkynyl group in accordance with the above definitions, for example alkyl-cycloalkyl, cycloalkylalkyl, alkylcycloalkenyl, alkenylcycloalkyl and alkynylcycloalkyl groups. An alkylcycloalkyl group preferably contains a cycloalkyl group that contains one or two ring systems having from 3 to 10 (preferably 3, 4, 5, 6 or 7) carbon atoms, and one or two alkyl, alkenyl or alkynyl groups having 1 or 2 to 6 carbon atoms, the cyclic groups being optionally substituted.

The expression heteroalkylcycloalkyl refers to alkylcycloalkyl groups as defined above in which one or more (preferably 1, 2 or 3) carbon atoms have been replaced each independently of the others by an oxygen, nitrogen, silicon, selenium, phosphorus or sulphur atom (preferably oxygen, sulphur or nitrogen). A heteroalkylcycloalkyl group preferably contains 1 or 2 ring systems having from 3 to 10 (preferably 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalkyl, alkynyl—hetero cyclo alkyl, hetero alkylcyclo alkyl, heteroalkylhetero cyclo alkyl and hetero-alkylheterocycloalkenyl, the cyclic groups being optionally substituted and saturated or mono-, di- or tri-unsaturated.

The expression aryl or Ar refers to an aromatic group that contains one or more rings containing from 6 to 14 ring carbon atoms, preferably from 6 to 10 (more preferably 6) ring carbon atoms. Examples are a phenyl, naphthyl, biphenyl, or indanyl group.

The expression heteroaryl refers to an aromatic group that contains one or more rings containing from 5 to 14 ring atoms, preferably from 5 to 10 (more preferably 5 or 6) ring atoms, and contains one or more (preferably 1, 2, 3 or 4) oxygen, nitrogen, phosphorus or sulphur ring atoms (preferably O, S or N). Examples are 2-pyridyl, 2-imidazolyl, 3-phenylpyr-rolyl, thiazolyl, oxazolyl, triazolyl, tetrazolyl, isoxazolyl, indazolyl, indolyl, benzimidazolyl, pyridazinyl, quinolinyl, purinyl, carbazolyl, acridinyl, pyrimidyl, 2,3′-bifuryl, 3-pyrazolyl and isoquinolinyl.

The expression aralkyl refers to a group containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylaryl-cycloalkyl and alkylarylcycloalkenyl groups. Specific examples of aralkyls are toluene, xylene, mesitylene, styrene, benzyl chloride, o-fluorotoluene, 1H-indene, tetralin, dihydronaphthalene, indanone, phenylcyclopentyl, cumene, cyclohexylphenyl, fluorene and indan. An aralkyl group preferably contains one or two aromatic ring systems (1 or 2 rings) containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (preferably 1, 2, 3 or 4) carbon atoms have been replaced each independently of the others by an oxygen, nitrogen, silicon, selenium, phosphorus, boron or sulphur atom (preferably oxygen, sulphur or nitrogen), that is to say to groups containing both aryl or heteroaryl and also alkyl, alkenyl, alkynyl and/or heteroalkyl and/or cycloalkyl and/or hetero—cycloalkyl groups in accordance with the above definitions. A heteroaralkyl group preferably contains one or two aromatic ring systems (1 or 2 rings) containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, 1, 2, 3 or 4 of those carbon atoms having been replaced each independently of the others by oxygen, sulphur or nitrogen atoms. Examples of heteroaralkyl groups are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, hetero-arylheterocycloalkyl, heteroarylheterocycloalkenyl, heteroarylalkylcycloalkyl, heteroarylalkyl-heterocycloalkenyl, heteroarylheteroalkylcycloalkyl, heteroarylheteroalkylcycloalkenyl, heteroalkylheteroarylalkyl and heteroarylheteroalkylheterocycloalkyl groups, the cyclic groups being saturated or mono- , di- or tri-unsaturated. Specific examples are a tetrahydroisoqui-nolinyl, benzoyl, 2- or 3-ethylindolyl, 4-methylpyridino, 2- , 3- or 4-methoxyphenyl, 4-ethoxy-phenyl, 2- , 3- or 4-carboxyphenylalkyl group.

The expression “halogen” or “halogen atom” as preferably used herein means fluorine, chlorine, bromine, or iodine.

The activity and more specifically the bioactivity of the compounds according to the present invention can be assessed using appropriate assays known to those skilled in the art, e.g. in vitro or in vivo assays. For instance, the antimicrobial activity may be determined using a standardized antibiotic assay^{[1]}, and the antiproliferative and/or cytotoxic activity may be determined via a standardized assay as described in reference [2].

Preferred is a compound of formula (I), or a pharmacologically acceptable salt thereof, wherein

R¹, R², R³ and R⁴ each independently represents H, OH, F, Cl, CF₃, NH₂, —C(O)OC₁₋₆ alkyl, —CONH₂, —OC₁₋₆ alkyl, —O—C(O)C₁₋₆ alkyl, —NHC₁₋₆ alkyl, —NH—C(O)C₁₋₆ alkyl; —C(═O)NHC₁₋₆ alkyl; —NHSO₂C₁₋₆ alkyl; SOCH₃; SOCF₃; —SO₂NH₂, SO₂N(C₁₋₆ alkyl); or a group comprising a linear or branched polyethylene glycol (PEG) group which comprises the formula —(CH₂—CH₂—O)ₜ— with t being an integer of from 2 to 100 and has a molecular weight (MW) of from 400 to 50000 Da;

R³¹ represents H or —C(═O)R; wherein R is —(CH₂)ₚ—X¹; and p and X¹ are defined as above.

In the compound of formula (I), R¹ and R² can each independently represent H, OH, F, Cl, CF₃, NH₂, —C(O)OC₁₋₆ alkyl, —CONH₂; —OC₁₋₆ alkyl; —NHC₁₋₆ alkyl; SOCH₃; SOCF₃; or —SO₂NH₂;

R³ or R⁴ can represent a group comprising a linear or branched polyethylene glycol (PEG) group which comprises the formula —(CH₂—CH₂—O)ₜ— with t being an integer of from 2 to 100 and has a molecular weight (MW) of from 400 to 50000 Da; and the other can be, independently of R¹, defined as R¹; and

R³¹ can represent H or —C(═O)R; wherein R is —(CH₂)ₚ—X¹; and p and X¹ are defined as above.

Further preferred is a compound of formula (I), or a pharmacologically acceptable salt thereof, wherein

R¹, R², R³ and R⁴ each independently represents OH, F, Cl, CF₃, NH₂, —C(O)OC₁₋₆ alkyl, —CONH₂, —OC₁₋₆ alkyl; SOCH₃; SOCF₃; or —SO₂NH₂;

R³¹ represents H or —C(═O)R; wherein R is —(CH₂)ₚ—X¹; and p and X¹ are defined as above.

Also preferred is a compound of formula (I), or a pharmacologically acceptable salt thereof, wherein R¹, R², R³ and R⁴ each independently represents OH, F, Cl, CF₃, NH₂, —OC₁₋₆ alkyl; or —SO₂NH₂;

R³¹ represents H or —C(═O)R; wherein R is —(CH₂)ₚ—X¹; and p and X¹ are defined as above

In the compound according to the present invention, R³¹ can represent H or a group

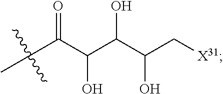

wherein X³¹ is OH or 0C_{1—3}alkyl.

The compound of formula (I) can be:

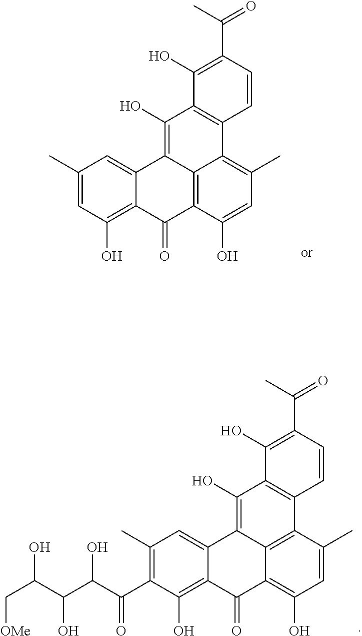

The therapeutic use of a compound of formula (I), its pharmacologically acceptable salt, solvate or hydrate and also a formulation and pharmaceutical composition which contain the same are within the scope of the present invention. The present invention also relates to the use of the compound of formula (I) as active ingredient in the preparation or manufacture of a medicament.

A pharmaceutical composition according to the present invention comprises at least one compound of formula (I) and, optionally, one or more carrier substance(s), excipient(s) and/or adjuvant(s). Pharmaceutical compositions may additionally comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives.

Furthermore, one or more other active ingredients may (but need not) be included in the pharmaceutical compositions provided herein. For instance, the compounds of the invention may advantageously be employed in combination with another antibiotic or antifungal agent, an anti-viral agent, an anti histamine, a non-steroidal anti-inflammatory drug, a disease modifying anti-rheumatic drug, another cytostatic drug, a drug with smooth muscle activity modulatory activity or mixtures of the aforementioned.

Pharmaceutical compositions may be formulated for any appropriate route of administration, including, for example, parenteral administration. The term parenteral as used herein includes subcutaneous, intradermal, intravascular such as, e.g., intravenous, intramuscular, spinal, intracranial, intrathecal, intraocular, periocular, intraorbital, intrasynovial and intraperitoneal injection, as well as any similar injection or infusion technique.

Carrier substances are, for example, cyclodextrins such as hydroxypropyl β-cyclodextrin, micelles or liposomes, excipients and/or adjuvants. Customary excipients include, for example, inert diluents such as, e.g., calcium carbonate, sodium carbonate, lactose, calcium phosphate or sodium phosphate, granulating and disintegrating agents such as, e.g., corn starch or alginic acid, binding agents such as, e.g., starch, gelatin or acacia, and lubricating agents such as, e.g., magnesium stearate or stearic acid. Examples of adjuvants are aluminum hydroxide, aluminum phosphate, calcium phosphate hydroxide, paraffin oil, squalene, thimerosal, detergents, Freund's complete adjuvant, or Freund's incomplete adjuvant.

For the prevention and/or treatment of bacterial infections, especially infections with Gram-positive bacteria, or proliferative diseases, the dose of the biologically active compound according to the invention may vary within wide limits and may be adjusted to individual requirements. Active compounds according to the present invention are generally administered in a therapeutically effective amount. The expression “therapeutically effective amount” denotes a quantity of the compound(s) that produces a result that in and of itself helps to ameliorate, heal, or cure the respective condition or disease. Preferred doses range from about 0.1 mg to about 140 mg per kilogram of body weight per day (about 0.5 mg to about 7 g per patient per day). The daily dose may be administered as a single dose or in a plurality of doses. The amount of active ingredient that may be combined with the carrier materials to produce a single dosage form will vary depending upon the host treated and the particular mode of administration. Dosage unit forms will generally contain between from about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient) and the severity of the particular disease undergoing therapy.

Preferred compounds of the invention will have certain pharmacological properties. Such properties include, but are not limited to oral bioavailability, such that the preferred oral dosage forms discussed above can provide therapeutically effective levels of the compound in vivo.

The compound according to the invention as well as the pharmaceutical composition according to the invention can be used as a medicament, which can be administered to a patient (e.g. parenterally to a human or an other mammal), and will be present within at least one body fluid or tissue of the patient. As used herein, the term “treatment” encompasses both disease-modifying treatment and symptomatic treatment, either of which may be prophylactic, i.e., before the onset of symptoms, in order to prevent, delay or reduce the severity of symptoms, or therapeutic, i.e., after the onset of symptoms, in order to reduce the severity and/or duration of symptoms. In particular, the conditions or diseases that can be ameliorated, prevented or treated using a compound of formula (I) or a pharmaceutical composition according to the invention include bacterial or fungal infections, or cancer. Accordingly, the present invention also provides methods for treating patients suffering from said diseases. Patients may include but are not limited to primates (especially humans), domesticated companion animals (such as dogs, cats, horses) and livestock (such as cattle, pigs, sheep), with dosages as described herein.

Within the present application the term “proliferative disease” encompasses disorders that are characterized by an overproduction (excessive proliferation) of a certain type of cells and turnover of cellular matrix, including cancer, atherosclerosis, rheumatoid arthritis, psoriasis, idiopathic pulmonary fibrosis, scleroderma and cirrhosis of the liver. For example, leukemia is a proliferative disorder characterized by an abnormal proliferation (overproduction) of white blood cells.

Within the present application the term “cancer” encompasses, but is not limited to, disorders, such as solid tumor cancer including breast cancer, lung cancer (non-small-cell lung cancer and small-cell lung cancer), prostate cancer, cancers of the oral cavity and pharynx (lip, tongue, mouth, pharynx), esophagus, stomach, small intestine, large intestine, colon, rectum, gallbladder and biliary passages, pancreas, larynx, lung, bone, osteosarcoma, connective tissue, skin cancer including Kaposi's syndrome, melanoma and skin metastasis, epidermoid cancer, basal cell carcinoma, cervix uteri, corpus endometrium, cancer of ovary, testis, bladder, ureter and urethra, kidney, eye, brain and central nervous system, pseudotumor cerebri, sarcoma, sarcoid, thyroid and other endocrine glands (including but not limited to carcinoid tumors), Hodgkin's disease, non-Hodkin's lymphomas, multiple myeloma, hematopoetic malignancies including leukemias and lymphomas including lymphocytic, granulocytic and monocytic lymphomas, tumor invasion, metastasis, ascites, tumor growth and angiogenesis.

Preferred compounds of the invention will have certain pharmacological properties. Such properties include, but are not limited to, bioavailability (especially with regard to oral administration), metabolic stability and sufficient solubility, such that the dosage forms can provide therapeutically effective levels of the compound in vivo.

The compounds of the present invention can be prepared in a number of ways well known to one skilled in the art of organic synthesis. The compounds of the present invention can be synthesized using methods known in the art of synthetic organic chemistry or by biotechnological approaches such as fermentation as appreciated by those skilled in the art. Since the structural complexity of the compounds precludes facile total synthetic access, semisynthetic as well as biotechnological approaches are commonly pursued in pharmaceutical research and development.

The following method for producing a compound of formula (I) also lies within the scope of the present invention. For instance, a compound of formula (I) can be produced by culturing Clostridium beijerinckii strain DSM13821. It is understood that the production of compounds of formula (I) is not limited to the use of the particular organism described herein, which is given for illustrative purpose only. The invention also includes the use of any mutants which are capable of producing a compound of formula (I) including natural mutants as well as artificial mutants, e.g. genetically manipulated mutants and the expression of the gene cluster responsible for biosynthesis in a producer strain or by heterologous expression in host strains.

A compound of formula (I) can be produced in liquid culture, by growing the respective microorganism under anaerobic conditions in media containing one or several different carbon sources, and one or different nitrogen sources. Also salts are essential for growth and production. Suitable carbon sources are different mono- , di- , and polysaccharides like maltose, glucose or carbon from amino acids like peptones. Nitrogen sources are ammonium, nitrate, urea, chitin or nitrogen from amino acids. The following inorganic ions support the growth or are essential in synthetic media: Mg-ions, Ca-ions, Fe-ions, Mn-ions, Zn-ions, K-ions, sulfate-ions, Cl-ions, phosphate-ions.

Temperatures for growth and production are between 25° C. to 40° C., preferred temperatures are between 30° C. and 38.50° C., especially at 37° C. The pH of the culture solution is from 5 to 8, preferably a pH of 5.9 to 6.1, more preferably a pH of 6.0.

A compound of formula (I) can also be obtained by chemical synthesis in a number of ways well known to one skilled in the art of organic synthesis using usual chemical reaction and synthesis methods.

### BRIEF DESCRIPTION OF THE FIGURES

FIG. 1. Structure of clostrubin A (1) and ³J (arrow) and ⁴J (dashed arrow) HMBC correlations. Observed enriched carbons from labeled acetate feeding experiments in ¹³C NMR spectra.

FIG. 2. (A) Structure of clostrubin B (2). (B) Observed ¹H-¹H COSY (bold lines) and HMBC (arrows) correlations of 2 in DMSO-d₆. (C) Observed ¹H-¹H COSY (bold lines) and HMBC (arrows) correlations of 2 in DMF-d₇.

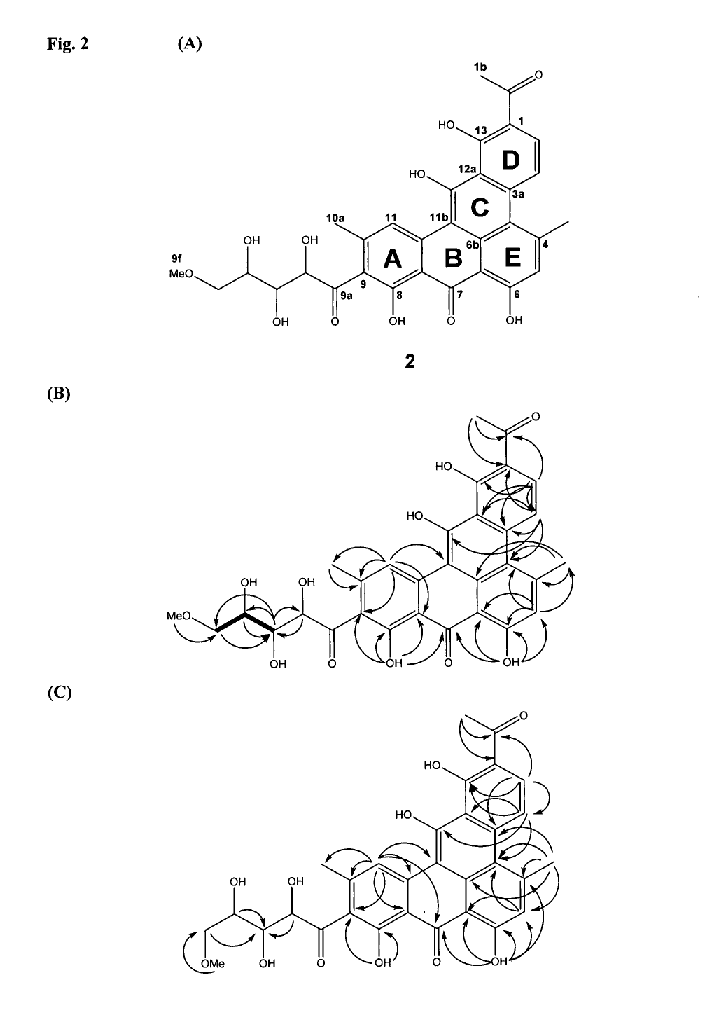

FIG. 3. (A) Diseased potato showing soft rot symptoms. (B) Diseased potato showing ring rot symptoms. (C) Diseased potato showing scab symptoms.

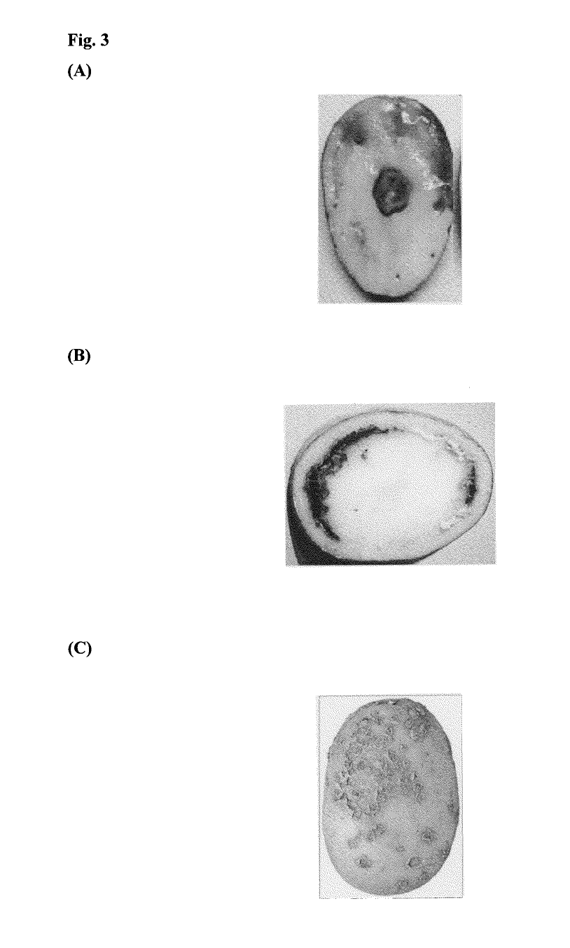

The present invention is now further illustrated by the following examples from which further features, embodiments and advantages of the present invention may be taken.

### EXAMPLES

### General Analytical Procedures

NMR spectra were measured on Bruker Avance III™ 600 MHz spectrometer with cryo probe in DMSO-d₆. Spectra were referenced to the residual solvent peak. UV spectra were obtained on a Shimadzu UV-1800 spectrometer. A Jasco Fourier Transform Infrared Spectrometer was used to measure the infrared spectra using the ATR technique. LC-HRMS measurements were carried out on a Thermo Fisher Scientific Exactive Orbitrap with an electrospray ion source using a Betasil 100-3 C₁₈ column (150×2.1 mm) and an elution gradient [solvent A: H₂O+0.1% HCOOH, solvent B: acetonitrile, gradient: 5% B for 1 min, 5% to 98% B in 15 min, 98% B for 15 min, flow rate: 0.2 mL min⁻¹, injection: 5 μL].

### Bacterial Strains and Culture Conditions

Clostridium beijerinckii strain DSM13821 was grown in either 2 ×YTG (16 g/L tryptone, 10 g/L yeast extract, 5 g/L NaCl and 5 g/L glucose) or P2 media anaerobically at 37° C.

C. beijerinckii strain DSM13821 was grown in batch culture in volumes ranging from 500 mL to 4 L in Biostat Q fermenters (B. Braun Biotech International) using either 2YTG or P2 media. Anaerobic conditions were maintained by sparging fermenters with N₂ for at least 3 hours prior to culture inoculation and during each fermentation run. 5% culture volume of a 24 hr old culture in the same media was used to inoculate each fermenter. Fermentation cultures were incubated at 37° C. with stirring at 200 rpm and pH 6.0 was maintained by automatic addition of 10% NaOH or 2 M HCl.

### Screening for Secondary Metbolites

Small scale bacterial cultures were grown in 50 mL volumes for 48 his and subsequently extracted with an equal volume of ethyl acetate overnight. The two phases were separated and the ethyl acetate phase was dried over Na₂SO₄ and concentrated in vacuo. The residue was dissolved in 0.5 mL methanol and was analyzed via analytical HPLC (Shimadzu LC-10Avp series with autosampler, high pressure pumps, column oven and DAD detector, C18 column (Nucleosil 100-5C18, 120×4.6 mm), 0.8 mL/min flow rate, gradient elution (MeCN/0.1% TFA 10/99.5 to 100/0 within 30 minutes).

### Example 1

### Clostrubin A Purification

Fermentation cultures (20 L) were initially extracted with equal volume of ethyl acetate overnight, after which the two phases were separated and the ethyl acetate phase was dried over Na₂SO₄ and then concentrated to dryness in vacuo. This extraction step, and all subsequent solvent extraction steps were performed three times each and subsequently combined. The dried phase was resuspended in 0.5M NaOH and then extracted with diethyl ether, which was subsequently removed and dried to give fraction 1 (F1). The pH of the aqueous phase was adjusted to 7.0 with HCl and again extracted with diethyl ether, which was then removed and dried to give F2. The aqueous phase was then acidified by the addition of HCl (to pH 2.0) and again extracted with diethyl ether, which was removed and dried to give F3. The acidic aqueous phase was subsequently extracted with ethyl acetate, which was removed and then dried, yielding F4. The aqueous phase was neutralized with NaOH (to pH 7.0) and again extracted with ethyl acetate, which was removed and dried yielding F5. Fraction F5 was then resuspended in 50% aqueous methanol, at which point particulates formed and the solution was filtered through a glass filter membrane. All particulate matter was removed from the filter by copious washing in MeOH, which was dried to completion. The sample was then analysed by HPLC and resuspended in DMSO-d₆ for NMR studies.

### Example 2

### Biophysical Characterisation of Clostrubin A (1)

Orange powder. HRESIMS: [M-H]⁻=413.1030 (calculated for C₂₅H₁₇O₆ 413.1031); UV λₘₐₓ =, (DMSO): 303 (ε76600), 377 (ε29600), 416 (ε14100), 551.0 (ε63000) nm; (dioxane), 230 (ε56000), 249 (ε65100), 307 (ε60300), 384 (ε24700), 426 (ε15000), 525 (ε53600) nm; (MeOH), 203 (ε88000), 248 (ε44900), 300 (ε41300), 377 (ε14000), 416 (ε7000), 542 (ε2700) nm; (pyridine), 378 (ε15400), 416 (ε7300), 554 (ε3200) nm; IR (ATR) : 1584, 1407, 1371, 1274, 1291, 1216, 1200, 997 cm⁻¹;

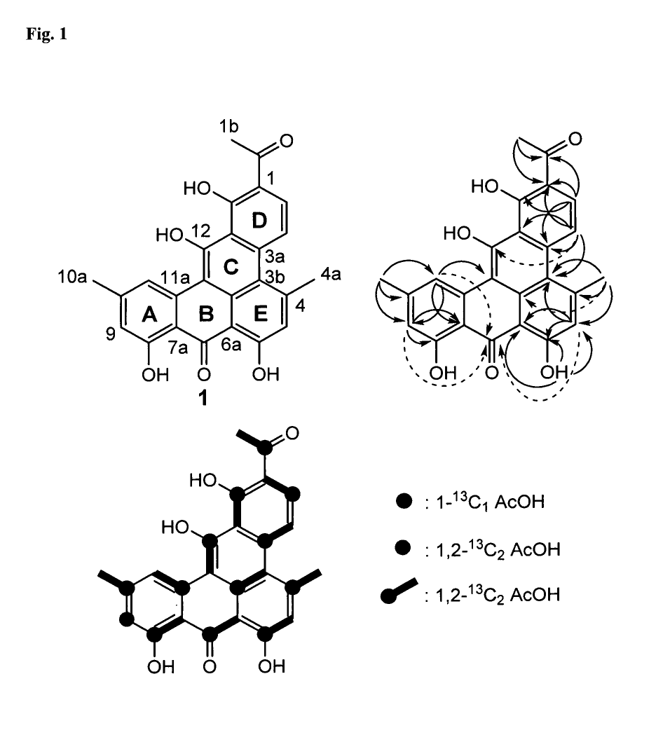

¹and ¹³C NMR spectra of isolated purple material indicated that 1 was an aromatic polyketide, as there were limited proton signals and many aromatic carbons. Detailed analysis of ¹H NMR spectrum indicated the presence of three methyl, five aromatic, and two phenolic protons. ¹³C NMR and DEPT135 spectra revealed three methyl, five aromatic methine, and 17 quaternary carbons, including four phenolic and two carbonyl carbons. Extensive HMBC and HSQC analyses showed three ring systems; ring A and E which are di- and tri-substituted cresols, respectively, and ring D which is a di-substituted 1-(2-hydroxyphenyl)ethan-1-one (FIG. 1). Ring A is connected to ring E by three ⁴J HMBC correlations from three singlet methine protons H5 (δ6.80), H9 (δ6.60), and H11 (δ9.30) to keto carbonyl carbon C7 (8 184.5). Two other ⁴J HMBC correlations from doublet methine proton H3 (δ7.60) to phenolic carbon C12 (δ178.0) and methyl proton H4a (δ2.90) to quaternary carbon C6b (δ133.6) suggested the presence of ring C. Finally, five aromatic rings were determined by two ³J HMBC correlations from H11 to C11b (δ133.6) and H3 to C3b (δ114.1). However, a quatenary carbon (C11a) was not assigned because of lacking HMBC correlations. To overcome this problem, cultures were grown in minimal media and supplemented with 1-¹³C or 1,2-¹³C labeled acetic acid, purified and analyzed by ¹³C NMR measurements. This process confirmed the presence of a number of quaternary carbons, whose correlations could now be reliably assigned to yield the final structure (1). The NMR data are summarized in Table 1 below.

TABLE 1
NMR spectroscopic data of clostrubin A (1) (¹H and ¹³C in
DMSO-d₆ at 300K).

| | | | | | 1,2-¹³C₂ AcOH | 1-¹³C AcOH |
|---|---|---|---|---|---|---|
| No. | ¹H (J, Hz) | | ¹³C (mult) | | (J_{CC}, Hz) | Enriched |
| 1 | | | 118.8 | (s) | 58 | −^{§} |
| 1a | | | 196.8 | (s) | 42 | +^{§} |
| 1b | 2.68 | (s) | 32.0 | (q) | 42 | − |
| 2 | 7.80 | (d 1.8) | 129.2 | (d) | 58 | + |
| 3 | 7.60 | (d 1.8) | 114.9 | (d) | 56 | − |
| 3a | | | 138.4 | (s) | 56 | + |
| 3b | | | 114.1 | (s) | 55 | − |
| 4 | | | 147.2 | (s) | 41 | + |
| 4a | 2.90 | (s) | 27.0 | (q) | 41 | − |
| 5 | 6.80 | (s) | 114.0 | (d) | 66 | − |
| 6 | 14.88 | (s, OH) | 164.5 | (s) | 66 | + |
| 6a | | | 107.3 | (s) | 58 | − |
| 6b | | | 133.6 | (s) | 55 | + |
| 7 | | | 184.7 | (s) | 58 | + |
| 7a | | | 111.2 | (s) | 62 | − |
| 8 | 13.70 | (s, OH) | 161.6 | (s) | 62 | + |
| 9 | 6.60 | (s) | 110.6 | (s) | + | − |
| 10 | | | 145.0 | (s) | 43 | + |
| 10a | 2.42 | (s) | 22.8 | (q) | 43 | − |
| 11 | 9.30 | (s) | 117.6 | (d) | 56 | + |

| | | | | |
|---|---|---|---|---|
| 11a | 138.6 | (s) | 56 | − |
| 11b | 104.3 | (s) | 66 | + |
| 12 | 177.9 | (s) | 66 | − |
| 12a | 117.3 | (s) | 59 | + |
| 13 | 167.4 | (s) | 59 | + |
| | 167.4 | (s) | 59 | + |

### Example 3

### Assessment of Antimicrobial Activity

The antibiotic activity of clostrubin A was tested using a standardized antibiotic assay^{[1]}. Clostrubin A (1) was found to be ineffective against the Gram-negative organism, Escherichia coli. However, 1 was shown to be superior to ciprofloxacin (Cip) at inhibiting the growth of several Gram-positive organisms, including the notorious hospital pathogens methicillin-resistant Staphylococcus aureus (MRSA) and vancomycin-resistant enterococci (VRE), with a minimum inhibitory concentration (MIC) of 0.05 μg/ml (0.12 μM) and 0.4 μg/ml (0.97 μM), respectively (Table 2). A similar antimicrobial activity as against MRSA was observed against Mycobacterium smegmatis (M smegmatis), Mycobacterium aurum (M aurum), Mycobacterium vaccae (M vaccae) and Mycobacterium fortuitum (M fortuitum)—see, Table 3.

TABLE 2
Antibacterial activity data for 1 and ciprofloxacin (cip).

| | MIC (μM) | | | |
|---|---|---|---|---|
| Compound | Ec | Bs | MRSA | VRE |
| 1 | >60.0 | 0.075 | 0.12 | 0.97 |
| Cip | 0.010 | 0.080 | 37 | 2.3 |

TABLE 3
Anti-mycobacterial activity data for 1 and ciprofloxacin (cip).

| | MIC (μM) | | | |
|---|---|---|---|---|
| Compound | M. smegmatis | M. aurum | M. vaccae | M. fortuitum |
| 1 | 0.48 | 0.12 | 0.12 | 0.12 |
| Cip | 4.71 | 0.30 | 1.20 | 0.60 |

### Example 4

### Assessment of Cytotoxic and Anti-Proliferative Activity

Antiproliferative and cytotoxic activity of clostrubin A was tested in a standardized assay^{[2]}. The results are shown in Table 4.

TABLE 4
Antiproliferative and cytotoxic activities of clostrubin A (1).

| | Antiproliferative Effect | | Cytotoxicity |
|---|---|---|---|
| | HUVEC GI₅₀ | K-562 GI₅₀ | HeLa CC₅₀ |
| 1 | 1.20 μM | 2.66 μM | 1.69 μM |

As can be taken from the above results, clostrubin A has moderate antiproliferative and cytotoxic activity.

### Example 5

### Isolation of Clostrubin B

A fermentation culture of C. beijerinckii strain DSM13821 (8 L; P2 medium) was extracted with EtOAc/dioxane (4 times) and the extracts were dried over sodium sulfate. After the solvent was removed under reduced pressure, the residue was dissolved in 2-propanol and then filtered through a glass filter. The precipitate was dissolved in DMSO and subjected to RP—HPLC (Nucleosil 100-5C18, 10×125 mm, flow rate 3.5 mL/min) using a gradient system: solvent A (H₂O containing 0.1% TFA); solvent B (acetonitrile); 30%B for 10 min, to 100%B for 35 min, to keep for 10 min, to yield 1.5 mg of clostrubin B.

### Example 6

### Biophysical Characterisation of Clostrubin B (2)

Orange powder. HRESIMS: [M-H]⁻=575.1570 (calculated for C₃₁H₂₇O₁₁ 575.1559); UV (DMSO) λₘₐₓ=288 (ε16100), 303 (ε16200), 379 (ε6300), 420 (ε4000), 555 (ε12100); IR (ATR) : 2924, 2855, 1576, 1456, 1374, 1234, 1059, 1023 cm⁻¹.

Although clostrubin B (2) has a similar UV spectrum to clostrubin A (1), HR—MS analysis revealed a molecular fomula of C₃₁H₂₈O₁₁ for 2, which is different from that of 1. The ¹H and ¹³C NMR specra of 2 in DMSO-d₆ indicated that the ring structure B-E is almost the same as that of 1. HSQC spectrum showed that methyl protons H10a (δ2.42) and an aromatic proton H9 (δ6.60) in A ring of 1 are disappeared in 2. However the HMBC correlation H11 (δ9.36) to C10a (δ22.2) revealed the presence of methyl group. Thus the structure of 2 was suggested to be modified at the position C9 (δ18.9). The elongated part at C9, which has a sugar feature from C9b to C9f, was confirmed by ¹H—¹H COSY (H9c to H9e) and HMBC (H9f/C9e, H9c/C9b, C9d, C9e) correlations. Because the HMBC correlations H11/C11a and H9b,c/C9a were not observed, NMR spectra of 2 were measured in DMF-d₇. The chemical shift at C11a was assigned as 138.6 ppm by the HMBC correlation H11 to C11a. In the HSQC spectrum of 2 in DMF-d₇, a correlation between an unusual broad methyl proton at H10a and C10a was also observed. However one carbonyl signal at C9a was not observed in both solvent conditions. Either weak or broad signals at positions 7a, 8, 9, 10, 10a, 11, and 11 a strongly indicated that a ring A structure in 2 may be tautomerized in both DMSO and DMF. Thus a carbon signal at C9a was not observed. The NMR data are summarized in Table 5 below.

TABLE 5
NMR spectroscopic data of clostrubin B (2) (¹H and ¹³C in DMF-d₇
and DMSO-d₆, respectively; at 300K).

| DMF-d₇ | | | | | DMSO-d₆ | | | |
|---|---|---|---|---|---|---|---|---|
| No. | ¹H (J, Hz) | | ¹³C (mult) | | ¹H (J, Hz) | | ¹³C (mult) | |
| 1 | | | 119.6 | (s) | | | 118.9 | (s) |
| 1a | | | 197.0 | (s) | | | 196.6 | (s) |
| 1b | 2.74 | (s) | 32.1 | (q) | 2.67 | (s) | 32.1 | (q) |
| 2 | 7.93 | (d 9.0) | 129.6 | (d) | 7.80 | (d 9.0) | 129.2 | (d) |
| 3 | 7.66 | (d 9.0) | 115.3 | (d) | 7.58 | (d 9.0) | 114.9 | (d) |
| 3a | | | 139.4 | (s) | | | 138.4 | (s) |
| 3b | | | 114.9 | (s) | | | 114.1 | (s) |
| 4 | | | 147.6 | (s) | | | 147.1 | (s) |
| 4a | 2.99 | (s) | 27.2 | (q) | 2.92 | (s) | 27.0 | (q) |
| 5 | 6.82 | (s) | 114.3 | (d) | 6.79 | (s) | 113.9 | (d) |
| 6 | 15.00 | (s, OH) | 165.5 | (s) | 14.88 | (s, OH) | 164.4 | (s) |
| 6a | | | 108.2 | (s) | | | 107.3 | (s) |
| 6b | | | 134.5 | (s) | | | 133.5 | (s) |
| 7 | | | 185.7 | (s) | | | 184.7 | (s) |
| 7a | | | 112.1 | (s) | | | 111.0 | (s) |
| 8 | 14.53 | (s, OH) | 161.7 | (s) | 14.35 | (s, OH) | 160.3 | (s) |
| 9 | | | 118.8 | (s) | | | 118.9 | (s) |
| 9a | | | — | | | | — | |
| 9b | 3.41 | (m) | 82.3 | (d) | 3.21 | (m) | 81.5 | (d) |
| 9c | 3.48 | (t 9.0) | 71.7 | (d) | 3.19 | (m) | 70.5 | (d) |
| 9d | 3.53 | (br) | 80.1 | (d) | 3.47 | (m) | 79.0 | (d) |
| 9e | 3.68 | (dd 12.1, 6.9) | 62.8 | (t) | 3.44 | (m) | 61.8 | (t) |
| | 3.89 | (dd 12.1, 2.1) | | | 3.72 | (brd 11.2) | | |
| 9f | 3.76 | (s) | 60.2 | (q) | 3.47 | (s) | 59.2 | (q) |
| 10 | | | 145.5 | (s) | | | 145.0 | (s) |
| 10a | 2.67 | (br) | 22.2 | (q) | 2.60 | (br) | 22.2 | (q) |
| 11 | 9.56 | (s) | 120.0 | (d) | 9.36 | (s) | 119.5 | (s) |
| 11a | | | 138.6 | (s) | | | 137.6 | (s) |
| 11b | | | 105.0 | (s) | | | 104.3 | (s) |
| 12 | | | 179.1 | (s) | | | 178.0 | (s) |
| 12a | | | 118.3 | (s) | | | 117.4 | (s) |
| 13 | | | 168.6 | (s) | | | 167.7 | (s) |

### Example 7

### Activity Against Potato Pathogens

The antimicrobial activity of clostrubin A (1) and clostrubin B (2) against the serious potato pathogens Bacillus pumilus (causing potato soft rot), Clavibacter michiganensis ssp. sepedonicus (causing potato ring rot), and Streptomyces scabies (causing potato scab) was tested. Both compounds were found to be more effective against these pathogens than ciprofloxacin. The results are shown in Table 6, where the minimum inhibitory concentration (MIC) for 1, 2, and ciprofloxacin against the above potato pathogens is indicated.

TABLE 6
Activity against potato pathogens

| | MIC (μM) | | |
|---|---|---|---|
| | | Clavibacter | |
| | | michiganensis | |
| | Bacillus pumilus | ssp. sepedonicus | Streptomyces scabies |
| Clostrubin A | 0.047 | 0.0947 | 0.0947 |
| Ciprofloxacin | 0.81 | 3.24 | 1.620 |
| Clostrubin B | 0.542 | 0.542 | 0.271 |

### REFERENCES

[1] I. Wiegand, K. Hilpert, R. E. Hancock, Nat. Protoc. 2008, 3, 163-175.

[2] M. Ziehl, J. He, H. M. Dahse, C. Hertweck, Angew. Chem., Int. Ed. 2005, 44, 1202-1205.

The features of the present invention disclosed in the specification, the claims and/or the drawings may both separately and in any combination thereof be material for realizing the invention in various forms thereof.

1. A compound of the general formula (I): 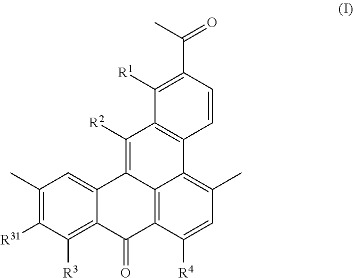
or a pharmacologically acceptable salt thereof, wherein
R¹, R², R³, R⁴ and R³¹ each independently represents H, OH, F, Cl, Br, I, NO₂, CF₃, CN, NH₂, NO₂, —(CH₂)ₘ—R; —C(═O)R; —C(═O)NHR; —NHC(═O)R; —NHSO₂R; S(O)ₙX²; SO₂NR⁵R⁶; or a group comprising a linear or branched polyethylene glycol (PEG) group which comprises the formula —(CH₂—CH₂—O)ₜ— with t being an integer of from 2 to 100 and has a molecular weight (MW) of from 400 to 50000 Da;
R represents H; —(CH₂)ₚ—X¹; —(CH₂)ₚ—OX¹; a C₁₋₆ heteroalkyl; a cycloalkyl; a heterocycloalkyl; an alkylcycloalkyl; a heteroalkylcycloalkyl; an aryl; a heteroaryl; an aralkyl; or a heteroaralkyl group;
R⁵ and R⁶ each independently represents a hydrogen atom; or —(CH₂)ₘ—R; or
R⁵ and R⁶ are taken together to form a 5- to 8-membered saturated, unsaturated or aromatic heterocycle containing 1 to 4 N atoms or 1 to 3 heteroatoms selected from the group consisting of nitrogen, oxygen and sulphur, which heterocycle may be unsubstituted or mono- , di or trisubstituted by halogen atom or R; or R⁵ and R⁶ are taken together to form a 5- to 8-membered saturated, unsaturated or aromatic heterocycle containing 1 to 3 heteroatoms selected from the group consisting of nitrogen, oxygen and sulphur, which is fused to one or two rings selected from the group consisting of cycloalkyl; heterocycloalkyl; alkylcycloalkyl; heteroalkylcycloalkyl; aryl; heteroaryl; aralkyl; and heteroaralkyl;
X¹ represents a C₁₋₆ alkyl, C₂₋₆ alkenyl, or C₂₋₆ alkynyl group, in which 1 to 5 H atoms may, independently of each other, be replaced by halogen atom, CN, CF₃, NO₂, OR or NHR, and/or in which one or two non-adjacent CH₂ group(s) may be replaced by O, NH, S, SO, SO2, or C3-7 cycloalkyl;
X² represents CN, CF₃, —(CH₂)ₚ—X¹, or NR⁵R⁶;
m is 0, 1, 2 or 3;
n is 0, 1 or 2; and
p is an integer from 0 to 6.

2. The compound according to claim 1, or a pharmacologically acceptable salt thereof, wherein
R¹, R², R³ and R⁴ each independently represents H, OH, F, Cl, CF₃, NH₂, —C(O)OC₁₋₆ alkyl, —CONH₂, —OC ₁₋₆ alkyl, —O—C(O)C₁₋₆ alkyl, —NHC₁₋₆ alkyl, —NH—C(O)C₁₋₆ alkyl; —C(═O)NHC₁₋₆ alkyl; —NHSO₂C₁₋₆ alkyl; SOCH₃, SOCF₃; —SO₂NH₂, SO₂N(C₁₋₆ alkyl); or a group comprising a linear or branched polyethylene glycol (PEG) group which comprises the formula —(CH₂—CH₂—O)ₜ— with t being an integer of from 2 to 100 and has a molecular weight (MW) of from 400 to 50000 Da;
R³¹ represents H or —C(═O)R; wherein R is —(CH₂)ₚ—X¹; and p and X¹ are defined as in claim 1.

3. The compound according to claim 1, or a pharmacologically acceptable salt thereof, wherein
R¹ and R² each independently represents H, OH, F, Cl, CF₃, NH₂, —C(O)OC₁₋₆ alkyl, —CONH₂; —OC₁₋₆ alkyl; —NHC₁₋₆ alkyl; SOCH₃, SOCF₃; or —SO₂NH₂;
R³ or R⁴ represents a group comprising a linear or branched polyethylene glycol (PEG) group which comprises the formula —(CH₂—CH₂—O)ₜ— with t being an integer of from 2 to 100 and has a molecular weight (MW) of from 400 to 50000 Da; and the other is, independently of R¹, defined as R¹;
R³¹ represents H or —C(═O)R; wherein R is —(CH₂)ₚ—X¹; and p and X¹ are defined as in claim 1.

4. The compound according to claim 1, or a pharmacologically acceptable salt thereof, wherein
R¹, R², R³ and R⁴ each independently represents OH, F, Cl, CF₃, NH₂, —C(O)OC₁₋₆ alkyl, —CONH₂, —OC₁₋₆ alkyl; SOCH₃; SOCF₃; or —SO₂NH₂;
R³¹ represents H or —C(═O)R; wherein R is —(CH₂)ₚ—X¹; and p and X¹ are defined as in claim 1.

5. The compound according to claim 1 or a pharmacologically acceptable salt thereof, wherein
R¹, R², R³ and R⁴ each independently represents OH, F, Cl, CF₃, NH₂, —OC₁₋₆ alkyl; or —SO₂NH₂;
R³¹ represents H or —C(═O)R; wherein R is —(CH₂)ₚ—X¹; and p and X¹ are defined as in claim 1.

6. The compound according to claim 1, wherein R³¹ represents H or a group 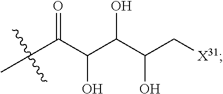
wherein X³¹ is OH or OC₁₋₃alkyl.

7. The compound according to claim 1, wherein the compound is:

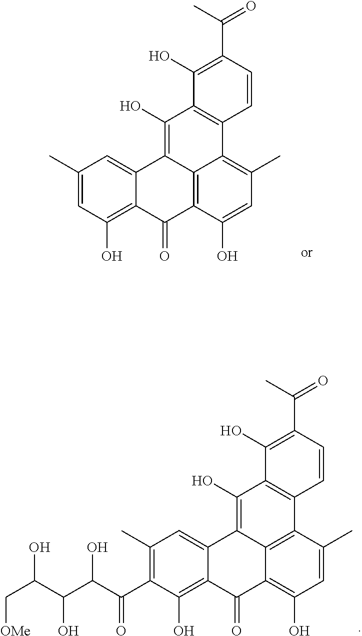

8. A pharmaceutical composition comprising at least one compound according to claim 1 and, optionally, one or more carrier substance(s), excipient(s) and/or adjuvant(s).

9. The compound according to claim 1, or the pharmaceutical composition for use as a medicament.

10. The compound according to claim 1 or the pharmaceutical composition, for use in the prevention and/or treatment of a bacterial infection.

11. The compound or the pharmaceutical composition for use as in claim 10, wherein the bacterial infection is caused by a Gram-positive microbe.

12. The compound according to claim 1, or the pharmaceutical composition for use in the prevention and/or treatment of a proliferative disease.

13. A method for the preparation of a compound of formula (I), the method comprising the steps of:
(a) culturing strain DSM 13821; and
(b) separating and retaining the compound from the culture broth, the step comprising precipitation on a glass filter.

14. A method for the treatment of a subject which is in need of such treatment, comprising the administration of a compound according to claim 1.

15. A method for the treatment of a subject which is in need of such treatment, comprising the administration of a pharmaceutical composition of claim 8.

16. A method for the treatment of a subject suffering from a bacterial infection, comprising administering an effective amount of a compound of claim 1 to the subject.

17. A method for the treatment of a subject suffering from a bacterial infection, comprising the administering an effective amount of a pharmaceutical composition of claim 8 to the subject.
